(19) United States
(12) Patent Application Publication
(10) Pub. No.: US 2024/0148833 A1
(43) Pub. Date: May 9, 2024

(54) COMPOSITION COMPRISING GLP-1 RECEPTOR AGONIST AND ACAT INHIBITOR

(71) Applicants: EFIL BioScience Inc., Daejeon (KR); Purdue Research Foundation, West Lafayette, IN (US)

(72) Inventors: Jeong-Hoon KIM, Daejeon (KR); Mu Lim CHOI, Daejeon (KR); Kee-Hong KIM, West Lafayette, IN (US)

(21) Appl. No.: 18/501,740

(22) Filed: Nov. 3, 2023

Related U.S. Application Data
(60) Provisional application No. 63/382,287, filed on Nov. 3, 2022.

(51) Int. Cl. A61K 38/26; A61K 31/18; A61P 3/04

(52) U.S. Cl. CPC A61K 38/26; A61K 31/18; A61P 3/04

(57) ABSTRACT

This present invention provides a pharmaceutical composition comprising one or more ACAT inhibitors and one or more GLP-1RAs. It also provides a method for controlling body weight comprising co-administering to a subject in need one or more ACAT inhibitors and one or more GLP-1RAs.

### CROSS-REFERENCE TO RELATED APPLICATION

The present application claims priority to U.S. Provisional Application No. 63/382,287 filed on Nov. 3, 2022, which is incorporated herein by reference.

### TECHNICAL FIELD

The present invention relates to compositions comprising one or more GLP-1 receptor agonists and one or more ACAT inhibitors and to methods for controlling body weight by co-administering one or more GLP-1 receptor agonists and one or more ACAT inhibitors.

### BACKGROUND

The hallmark of obesity is fat mass enlargement, resulting from an increase in adipocyte cell size and number. In hypertrophied adipocytes, free cholesterol has an augmented uptake, leading to its accumulation in lipid droplets in proportion to increased triglyceride content. B. R. Krause, A. D. Hartman, Adipose tissue and cholesterol metabolism, J Lipid Res 25 (1984) 97-110; F. T. Doole, T. Kumarage, R. Ashkar, M. F. Brown, Cholesterol Stiffening of Lipid Membranes, J Membr Biol. 255 (2022) 385-405, which is incorporated herein by reference. 10.1007/s00232-022-00263-9. Acyl-coenzyme A:cholesterol acyltransferase (ACAT) that catalyzes the conversion of free cholesterol to cholesteryl ester (CE) using adenosine triphosphate and coenzyme A plays an important role in cellular cholesterol storage. S. Mukherjee, G. Kunitake, R. B. Alfin-Slater, The esterification of cholesterol with palmitic acid by rat liver homogenates., J Biol Chem 230 (1958) 91-96, which is incorporated herein by reference. Increased ACAT1 expression is shown to be associated with increased adiposity and adipogenesis in vitro. Y. Zhu, C. Y. Chen, J. Li, J. X. Cheng, M. Jang, K. H. Kim, In vitro exploration of ACAT contributions to lipid droplet formation during adipogenesis, J Lipid Res 59 (2018) 820-829. 10.1194/jlr.M081745; Y. Xu, X. Du, N. Turner, A. J. Brown, H. Yang, Enhanced acyl-CoA:cholesterol acyltransferase activity increases cholesterol levels on the lipid droplet surface and impairs adipocyte function, J Biol Chem 294 (2019) 19306-19321, which are incorporated herein by reference. Inhibition of ACAT activity was reported to suppress lipid droplet formation and expansion during adipogenesis in vitro. Y. Zhu, C. Y. Chen, J. Li, J. X. Cheng, M. Jang, K. H. Kim, In vitro exploration of ACAT contributions to lipid droplet formation during adipogenesis, J Lipid Res 59 (2018) 820-829, which is incorporated herein by reference. Additionally, it was recently reported that ACAT inhibitor induced significant weight loss while concurrently suppressing food intake in diet-induced obese (DIO) mice with lower blood levels of markers associated with obesity and insulin resistance. Y. Zhu, S. Q. Kim, Y. Zhang, Q. Liu, K. H. Kim, Pharmacological inhibition of acyl-coenzyme A:cholesterol acyltransferase alleviates obesity and insulin resistance in diet-induced obese mice by regulating food intake, Metabolism 123 (2021) 154861, which is incorporated herein by reference. It was reported that an ACAT inhibitor can lower body weight and food intake. U.S. patent application Ser. No. 16/461,597, which is incorporated herein by reference.

Glucagon-like peptide-1 receptor agonists (GLP-1 RAs) including semaglutide and liraglutide were reported to lower body weight and treat type 2 diabetes. Diabetes Ther. 2020 Sep.; 11(9): 1965-1982; U.S. Pat. Nos. 8,129,343, 9,993,430, which are incorporated herein by reference. A series of clinical trials named Semaglutide Treatment Effect in People with obesity (STEP) have successfully shown 14.9%-17.4% weight loss effect by once-weekly sc injection of semaglutide 2.4 mg after 68 weeks. T. A. Wadden, T. S. Bailey, L. K. Billings, M. Davies, J. P. Frias, A. Koroleva, I. Lingvay, P. M. O'Neil, D. M. Rubino, D. Skovgaard, S. O. R. Wallenstein, W. T. Garvey, Effect of Subcutaneous Semaglutide vs Placebo as an Adjunct to Intensive Behavioral Therapy on Body Weight in Adults With Overweight or Obesity: The STEP 3 Randomized Clinical Trial, JAMA 325 (2021) 1403-1413. 10.1001/jama.2021.1831.; D. Rubino, N. Abrahamsson, M. Davies, D. Hesse, F. L. Greenway, C. Jensen, I. Lingvay, O. Mosenzon, J. Rosenstock, M. A. Rubio, G. Rudofsky, S. Tadayon, T. A. Wadden, D. Dicker, Effect of Continued Weekly Subcutaneous Semaglutide vs Placebo on Weight Loss Maintenance in Adults With Overweight or Obesity: The STEP 4 Randomized Clinical Trial, JAMA 325 (2021) 1414-1425. 10.1001/jama.2021.3224, which are incorporated herein by reference. The success of semaglutide in treating obesity, resulting in double-digit percentage of body weight loss, has led to its clinical approval. However, concerns have arisen regarding the dose-dependent gastrointestinal events associated with semaglutide and the immediate weight regain experienced after discontinuation of the treatment. These concerns have prompted the exploration of new semaglutide regimens that can offer even greater efficacy and improved tolerability.

This BACKGROUND section introduces aspects that may help facilitate a better understanding of the disclosure. Accordingly, these statements are to be read in this light and are not to be understood as admissions about what is or is not prior art.

### SUMMARY

An aspect of the present invention provides a method for controlling body weight comprising co-administering to a subject in need a therapeutically effective amount of one or more ACAT inhibitors or pharmaceutically acceptable salts thereof and a therapeutically effective amount of one or more GLP-1 RAs or pharmaceutically acceptable salts thereof.

In some embodiments, the GLP-1 RA may be selected from the group consisting of lixisenatide, liraglutide, exenatide, exenatide extended release, albiglutide, semaglutide, ITCA 650, dulaglutide, tirzepatide, retatrutide, orforglipron, lotiglipron, efpeglenatide, and taspoglutide.

In some embodiments, the ACAT inhibitor may be selected from the group consisting of avasimibe (CI-1011), CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511), E5324, FR145237, CL277,082, YM-17E, FR129169, K-604, pyrocarbonate, beauveriolides I, and methanol extracts of Saururus chinensis root containing saucerneol B and manassantin B.

In some embodiments, the GLP-1 RA and the ACAT inhibitor may be formed in a formulation and the formulation is administered.

In some embodiments, the GLP-1 RA and the ACAT inhibitor may be formulated separately and administered at the same time or sequentially.

In some embodiments, the GLP-1 RA may be selected from the group consisting of semaglutide, liraglutide, and tirzepatide, and the ACAT inhibitor may be avasimibe or a pharmaceutically acceptable salt thereof.

Another aspect of the present invention provides a composition comprising: an ACAT inhibitor or a pharmaceutically acceptable salt thereof; a GLP-1 RA or a pharmaceutically acceptable salt thereof; and a pharmaceutically acceptable excipient or carrier.

In some embodiments, the GLP-1 RA may be selected from the group consisting of lixisenatide, liraglutide, exenatide, exenatide extended release, albiglutide, semaglutide, ITCA 650, dulaglutide, tirzepatide, retatrutide, orforglipron, lotiglipron, efpeglenatide, and taspoglutide.

In some embodiments, the ACAT inhibitor may be selected from the group consisting of avasimibe (CI-1011), CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511), E5324, FR145237, CL277,082, YM-17E, FR129169, K-604, pyrocarbonate, beauveriolides I, and methanol extracts of Saururus chinensis root containing saucerneol B and manassantin B.

In some embodiments, the GLP-1 RA may be selected from the group consisting of semaglutide, liraglutide, and tirzepatide, and the ACAT inhibitor may be avasimibe or a pharmaceutically acceptable salt thereof.

Each embodiment disclosed herein is contemplated as being applicable to each of the other disclosed embodiments. Thus, all combinations of the various elements described herein are within the scope of the invention.

### BRIEF DESCRIPTION OF THE DRAWINGS

FIG. 1 shows combined effects of semaglutide and avasimibe in morbidly obese male mice after daily sc administration for 28 days. A, Study design: obese male C57BL/6 mice received semaglutide (0.04 mg/kg body weight) or vehicle solution in the presence or absence of avasimibe (10 mg/kg body weight) for the indicated days. B, Body weight. C, % body weight change at day 28. D, Food intake. E, Relative organ weight. All values are presented as mean±SEM; n=10 mice/group. Multiple comparisons were performed by one-way ANOVA with Tukey post hoc test (C), or two-way ANOVA with Tukey post hoc test (B, D); *p<0.05, ** p<0.01, ***p<0.001 and ****p<0.0005 significant vs. Vehicle group: \hat{ }p<0.05, \hat{ }\hat{ }p<0.01 and \hat{ }\hat{ }\hat{ }p<0.001 significant vs. Semaglutide group. Student's t-test was employed to post-sacrifice 1 in B and D, and E: § p<0.05, §§ p<0.01 and § § § § p<0.0005. EpiWAT, epididymal white adipose tissue; PRAT, perirenal adipose tissue; RetroWAT, retroperitoneal white adipose tissue.

FIG. 2 shows combined effects of semaglutide and avasimibe in moderately obese male mice after once every three days sc administration for 23 days. A, Study design: obese male C57BL/6 mice received semaglutide (0.04 mg/kg body weight) or vehicle solution in the presence or absence of avasimibe (20 mg/kg body weight) every three days. B, Body weight. Arrows indicate days of the drug administration. C, % body weight change at sacrifice. D, Daily food intake. E, Cumulative food intake. F, G, OGTT result and area under the curve. Time 0 is immediately before glucose challenge. All values are presented as mean±SEM; n=5-6 mice/group. Multiple comparisons were performed by one-way ANOVA with Tukey post hoc test (C, E, F and G), or two-way ANOVA with Tukey post hoc test (B, D); *p<0.05, ** p<0.01, ***p<0.001 and ****p<0.0005 significant vs. Vehicle group.

FIG. 3 shows combined effect of semaglutide and avasimibe in fat mass and adipocyte size in moderately obese male mice after once every three days sc administration for 23 days. A, Body composition. B, Relative organ weight. C, PCC between body weight and lean mass. D, PCC between body weight and fat mass. E, PCC between ingWAT and fat mass. F, IngWAT H&E staining (scale bar: 50 μm). G, Adipocyte size distribution in IngWAT. All values are presented as mean±SEM; n=5-6 mice/group. Multiple comparisons were performed by one-way ANOVA with Tukey post hoc test (A, B, and G). *p<0.05, ** p<0.01, and ***p<0.001 significant vs. Vehicle group. EpiWAT, epididymal white adipose tissue; IngWAT, inguinal white adipose tissue; RetroWAT, retroperitoneal white adipose tissue.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure the term “about” can allow for a degree of variability in a value or range, for example, within 10%, within 5%, or within 1% of a stated value or of a stated limit of a range.

In the present disclosure the term “substantially” can allow for a degree of variability in a value or range, for example, within 90%, within 95%, or within 99% of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

As used herein, the term “GLP-1 RA” means a glucagon-like peptide 1 receptor agonist. Non-limiting examples of the GLP-1 RA include lixisenatide, liraglutide, exenatide, exenatide extended release, albiglutide, semaglutide, ITCA 650, dulaglutide, tirzepatide, retatrutide, orforglipron, lotiglipron, efpeglenatide, and taspoglutide.

As used herein, the term “ACAT inhibitor” means a small or large molecule that can inhibit ACAT activity. Non-limiting examples of the ACAT inhibitor include avasimibe (CI-1011), K-604, CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511) and F12511 analogs (analogs 1, 2, 2c and 3 or F26) (US2006/0135785), E5324, FR145237, CL277,082, YM-17E, FR129169, diethyl pyrocarbonate (Cho, et al. 2003, Biochem. Biophys. Res. Comm. 309:864-872), beauveriolides I and beauveriolides III (Oshiro, et al. 2007, J. Antibiotics 60:43-51), beauveriolide analogs (258, 274, 280, 285 and 301) (Tomoda & Doi, 2008, Accounts Chem. Res. 41:32-39), Compound 1A and its derivatives (1B, 1C and 1D) (Lada, et al. 2004, J. Lipid Res. 45:378-386), methanol extracts of Saururus chinensis root containing saucerneol B and manassantin B (Lee, et al. 2004, Bioorg. Med. Chem. Lett. 14:3109-3112), and derivatives of anilidic, ureidic or diphenyl imidazole compounds (PCT/US2014/054917).

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fishes and the like.

As used herein, the term “administration” or “administering” of the subject compound refers to providing a compound of the invention and/or a prodrug thereof to a subject in need of treatment.

As used herein, the term “effective amount” or “therapeutically effective amount” refer to a sufficient amount of an active ingredient(s) described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose-escalation study. By way of example only, a therapeutically effective amount of a compound of the invention may be in the range of e.g., about 0.01 mg/kg/day to about 1000 mg/kg/day, from about 0.1 mg/kg/day to about 500 mg/kg/day, from about 0.1 mg (×2)/kg/day to about 500 mg (×2)/kg/day.

In addition, such compounds and compositions may be administered singly or in combination with one or more additional therapeutic agents. The methods of administration of such compounds and compositions may include, but are not limited to, intravenous administration, inhalation, oral administration, rectal administration, parenteral, intravitreal administration, subcutaneous administration, intramuscular administration, intranasal administration, dermal administration, topical administration, ophthalmic administration, buccal administration, tracheal administration, bronchial administration, sublingual administration or optic administration. Compounds provided herein may be administered by way of known pharmaceutical formulations, including tablets, capsules or elixirs for oral administration, suppositories for rectal administration, sterile solutions or suspensions for parenteral or intramuscular administration, lotions, gels, ointments or creams for topical administration, and the like. In some embodiments, such pharmaceutical compositions are formulated as tablets, pills, capsules, a liquid, an inhalant, a nasal spray solution, a suppository, a solution, a gel, an emulsion, an ointment, eye drops, or ear drops.

The therapeutically effective amount may vary depending on, among others, the disease indicated, the severity of the disease, the age and relative health of the subject, the potency of the compound administered, the mode of administration and the treatment desired. The required dosage will also vary depending on the mode of administration, the particular condition to be treated and the effect desired.

The compounds described herein include all stereoisomers, geometric isomers, tautomers, isotopes, and prodrug of the structures depicted. The compounds described herein can be present in various forms including crystalline, powder and amorphous forms of those compounds, pharmaceutically acceptable salts, including, for example, polymorphs, pseudopolymorphs, solvates, hydrates, unsolvated polymorphs (including anhydrates), conformational polymorphs, and amorphous forms of the compounds, as well as mixtures thereof.

As used herein, the term “pharmaceutically acceptable” material refers a material, such as a carrier or diluent, which does not abrogate the biological activity or properties of the compounds described herein. Such materials are administered to an individual without causing undesirable biological effects or interacting in a deleterious manner with any of the components of the composition in which it is contained.

As used herein, the term “pharmaceutically acceptable salt” refers to a formulation of a compound that does not cause significant irritation to an organism to which it is administered and does not abrogate the biological activity and properties of the compounds described herein.

Pharmaceutically acceptable salt forms may include pharmaceutically acceptable acidic/anionic or basic/cationic salts (UK Journal of Pharmaceutical and Biosciences Vol. 2(4), 01-04, 2014, which is incorporated herein by reference). Pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphosphate, polygalacturonate, salicylate, stearate, subacetate, succinate, sulfate, hydrogensulfate, tannate, tartrate, teoclate, tosylate, and triethiodide salts. Pharmaceutically acceptable basic/cationic salts include, the sodium, potassium, calcium, magnesium, diethanolamine, N-methyl-D-glucamine, L-lysine, L-arginine, ammonium, ethanolamine, piperazine, and triethanolamine salts.

A pharmaceutically acceptable acid addition salt of a compound of the invention may be prepared by methods known in the art and may be formed by reaction of the free base form of the compound with a suitable inorganic or organic acid including, but not limited to, hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, and hexanoic acid. A pharmaceutically acceptable acid addition salt can comprise or be, for example, a hydrobromide, hydrochloride, sulfate, bisulfate, nitrate, phosphate, succinate, maleate, formarate, acetate, propionate, fumarate, citrate, tartrate, lactate, benzoate, carbonate, benzathine, chloroprocaine, choline, histidine, meglumine, meglumine, procaine, triethylamine, besylate, decanoate, ethylenediamine, salicylate, glutamate, aspartate, p-toluenesulfonate, benzenesulfonate, methanesulfonate, ethanesulfonate, naphthalenesulfonate (e.g., 2-naphthalenesulfonate), and hexanoate salt.

A pharmaceutically acceptable base addition salt of a compound of the invention may also be prepared by methods known in the art and may be formed by the reaction of the free base form of the compound with a suitable inorganic or organic base including, but not limited to, hydroxide or other salt of sodium, potassium, lithium, ammonium, calcium, magnesium, iron, zinc, copper, manganese, aluminum, tromethamine, glycolate, hydrabamine, methylbromide, methylnitrate, octanoate, oleate, and the like.

A free acid or free base form of a compound of the invention may be prepared by methods known in the art (e.g., for further details see L. D. Bigley, S. M. Berg, D. C. Monkhouse, in “Encyclopedia of Pharmaceutical Technology”. Eds, J. Swarbrick and J. C. Boylam, Vol 13, Marcel Dekker, Inc., 1995, pp. 453-499, which is incorporated herein by reference). For example, a compound of the invention in an acid addition salt form may be converted to the corresponding free base form by treating with a suitable base (e.g., ammonium hydroxide solution, sodium hydroxide, and the like). A compound of the invention in a base addition salt form may be converted to the corresponding free acid by treating with a suitable acid (e.g., hydrochloric acid, etc.).

Aspects of this disclosure include prodrug forms of any of the compounds described herein. Any convenient prodrug forms of the subject compounds can be prepared, for example, according to the strategies and methods described by Rautio et al. (“Prodrugs: design and clinical applications”, Nature Reviews Drug Discovery 7, 255-270 (February 2008)).

Prodrug derivatives of the compounds of the invention may be prepared by methods known to those of ordinary skill in the art (e.g., for further details see Saulnier et al., Bioorg. Med. Chem. Letters, 1994, 4, 1985, which is incorporated herein by reference). Protected derivatives of the compounds of the invention may be prepared by means known to those of ordinary skill in the art. A detailed description of techniques applicable to the creation of protecting groups and their removal can be found in T. W. Greene, “Protecting Groups in Organic Chemistry,” 3rd edition, John Wiley and Sons, Inc., 1999 and “Design of Prodrugs”, ed. 11. Bundgaard, Elsevier, 1985, which are incorporated herein by reference.

The compounds of the present disclosure may be prepared as stereoisomers. Where the compounds have at least one chiral center, they may exist as enantiomers. Where the compounds possess two or more chiral centers, they may exist as diastereomers. The compounds of the invention may be prepared as racemic mixtures. Alternatively, the compounds of the invention may be prepared as their individual enantiomers or diastereomers by reaction of a racemic mixture of the compound with an optically active resolving agent to form a pair of diastereo-isomeric compounds, separating the diastereomers, and recovering the optically pure enantiomers. Resolution of enantiomers may be carried out using covalent diastereomeric derivatives of the compounds of the invention, or by using dissociable complexes (e.g., crystalline diastereomeric salts). Diastereomers have distinct physical properties (e.g., melting points, boiling points, solubility, reactivity, etc.) and may be readily separated by taking advantage of these dissimilarities. The diastereomers may be separated by chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet and Samuel H. Wilen, “Enantiomers, Racemates and Resolutions” John Wiley And Sons, Inc., 1981, which is incorporated herein by reference.

The compounds of the invention may be prepared as solvates (e.g., hydrates). The term “solvate” refers to a complex of variable stoichiometry formed by a solute (for example, a compound of the invention or a pharmaceutically acceptable salt thereof) and a solvent. Such solvents for the purpose of the invention may not interfere with the biological activity of the solute. Non-limiting examples of suitable solvents include water, acetone, methanol, ethanol and acetic acid. Preferably the solvent used is a pharmaceutically acceptable solvent.

Furthermore, the compounds of the invention may be prepared as crystalline forms. The crystalline forms may exist as polymorphs.

It should be noted that in view of the close relationship between the compound of the invention and their other forms, whenever a compound is referred to in this context herein, a corresponding salt, diastereomer, enantiomer, racemate, crystalline, polymorph, prodrug, hydrate, or solvate is also intended, if it is possible or appropriate under certain circumstances.

Another aspect of the present invention provides a composition for controlling body weight. The composition comprises a therapeutically effective amount of one or more ACAT inhibitors and a therapeutically effective amount of one or more GLP-1 RAs.

As used herein, the term “composition” is intended to encompass a product comprising the compound, salt, diastereomer, enantiomer, racemate, hydrate, solvate, or a pharmaceutical combination thereof in the therapeutically effective amount, as well as any other product which results, directly or indirectly, from claimed compound, salt, diastereomer, enantiomer, racemate, hydrate, solvate, or a pharmaceutical combination thereof.

As used herein, the term “pharmaceutical composition” refers to a mixture of a therapeutically active component (ingredient) with one or more other components, which may be chemically or biologically active or inactive. Such components may include, but not limited to, carriers, stabilizers, diluents, dispersing agents, suspending agents, thickening agents, excipients, and adjuvants.

As used herein, the term “pharmaceutical combination” means a product that results from the mixing or combining of more than one therapeutically active ingredient.

As used herein, the term “acceptable” with respect to a formulation, composition or ingredient, as used herein, means having no persistent detrimental effect on the general health of the subject being treated.

As used herein, the term “carrier” refers to chemical or biological material that can facilitate the incorporation of a therapeutically active ingredient(s) into cells or tissues.

Suitable excipients may include, for example, water, pharmaceutically acceptable organic solvents such as paraffins (e.g., petroleum fractions), vegetable oils (e.g. groundnut or sesame oil), mono- or polyfunctional alcohols (e.g., ethanol or glycerol), carriers such as natural mineral powders (e.g., kaoline, clays, talc, chalk), synthetic mineral powders (e.g., highly dispersed silicic acid and silicates), sugars (e.g., cane sugar, lactose and glucose), emulsifiers (e.g., lignin, spent sulphite liquors, methylcellulose, starch and polyvinylpyrrolidone), and lubricants (e.g., magnesium stearate, talc, stearic acid and sodium lauryl sulphate).

Any suitable pharmaceutically acceptable carriers, stabilizers, diluents, dispersing agents, suspending agents, thickening agents, excipients, and adjuvants known to those of ordinary skill in the art for use in pharmaceutical compositions may be selected and employed in the compositions described herein. The compositions described herein may be in the form of a solid, liquid, or gas (aerosol). For example, they may be in the form of tablets (coated tablets) made of, for example, collidone or shellac, gum Arabic, talc, titanium dioxide or sugar, capsules (gelatin), solutions (aqueous or aqueous-ethanolic solution), syrups containing the active substances, emulsions or inhalable powders (of various saccharides such as lactose or glucose, salts and mixture of these excipients with one another), and aerosols (propellant-containing or-free inhale solutions). Also, the compositions described herein may be formulated for sustained or slow release.

Other embodiments and uses will be apparent to one skilled in the art in light of the present disclosures. The following examples are provided merely as illustrative of various embodiments and shall not be construed to limit the invention in any way.

### EXAMPLES

### Example 1: Materials and Methods

1. Animal Husbandry and Administration of Drugs

Cohort 1: The first set of animal study was conducted at Nonclinical Research Institute, ChemOn Inc., and experimental procedures were approved by Institutional Animal Care and Use Committee (serial number 2021-09-007). Eight-week-old male C57BL/6 mice were purchased (Orient Bio Inc., Republic of Korea). After 2-week acclimation, the mice were fed with high-fat (HF) diet (cat #: D12492, Research Diets Inc.) for 12 weeks to develop DIO. These mice were then randomly assigned to control, avasimibe, semaglutide, and combination groups. Avasimibe solution was prepared for making the final concentration of avasimibe at 10 mg/kg BW, as described in Y. Zhu, S. Q. Kim, Y. Zhang, Q. Liu, K. H. Kim, Pharmacological inhibition of acyl-coenzyme A:cholesterol acyltransferase alleviates obesity and insulin resistance in diet-induced obese mice by regulating food intake, Metabolism 123 (2021) 154861, which is incorporated herein by reference. Semaglutide stock solution prepared in DMSO (1 mg/ml) was diluted with PBS for the final concentration of 0.04 mg/kg BW. The drug solution or the vehicle solution at a volume of 10 μl/g BW was injected to the mice sc daily for the indicated days.

Cohort 2: The second set of animal experiments were conducted according to a protocol approved by the Purdue Animal Care and Use Committee (protocol number: 11129000347). Male C57BL/6 DIO mice were purchased from Jackson Laboratory. After 8-week HF diet feeding, mice were randomly assigned to the same groups as cohort 1: control, avasimibe, semaglutide, and combination groups. Avasimibe (20 mg/kg BW) and semaglutide (0.04 mg/kg BW) were prepared in the method used with regard to Cohort 1, and the drugs were sc injected every three days for 23 days. Mice were kept on a 12/12 h light/dark cycle in a humidity and temperature control facility with ad libitum access to food and water.

2. Body Composition and Blood Analysis

Body composition (e.g., fat mass, lean mass, free water, and total water) changes were assessed right before sacrificing the second cohort of mice using EchoMRI (Echo Medical Systems). Blood samples were obtained after fasting for 3h, frozen and stored until analysis. Aspartate transaminase (AST), alanine aminotransferase (ALT), glucose, triglycerides (TG), high-density lipoprotein (HDL-C) and low-density lipoprotein cholesterol (LDL-C) were also measured (AU680 Chemistry Analyzer, Beckman Coulter). The levels of serum leptin (cat #: ab100718, Abcam) and insulin (cat #: ab277390, Abcam) were determined by enzyme-linked immunosorbent assay kit and cholesterol/CE (cat #: ab65359, Abcam) was determined by an enzymatic assay according to the manufacturers' instructions.

3. Oral Glucose Tolerance Test (OGTT)

Mice were fasted for 6 hrs (07:00-13:00) prior to oral administration of D-glucose (2 g/kg BW). Tail blood samples were obtained before (at 0) and at 15, 30, 60, 90 and 120 minutes after glucose challenge for the measurement of blood glucose concentration using a CONTOUR® NEXT glucometer (Ascensia Diabetes Care, Parsippany, NJ, USA).

4. Tissue Harvest and Histological Measurement of Adipocyte Size

During animal sacrifice, adipose tissues were rinsed with saline solution, fixed in 10% neutral formalin buffered solution, and embedded in paraffin. The tissues were then cut into 4 m sections and stained with hematoxylin and eosin (H&E) to measure cell size (˜100 cells/mouse) by ImageJ software (NIH).

5. Statistical Analysis

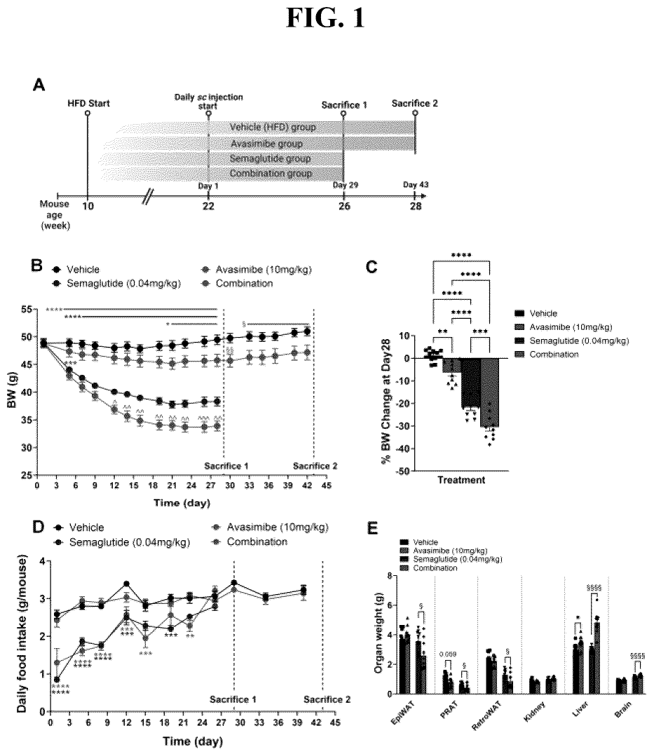

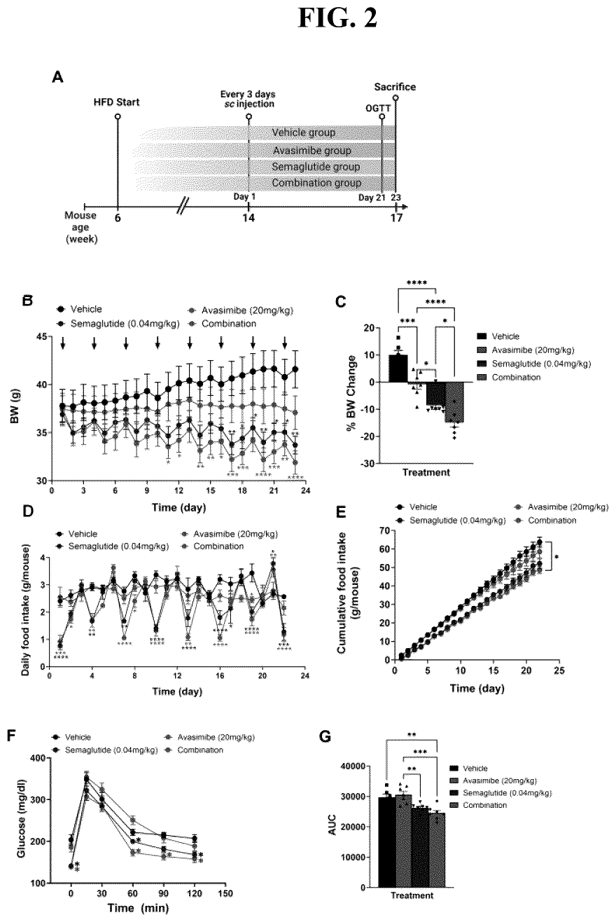

Data were presented as mean±SEM, and analyses were performed using GraphPad Prism 9.4.1 (GraphPad). Comparisons between the experimental and control groups were performed by one-way ANOVA (FIGS. 1C, 2C, 2E, 2F 2G, 3A, 3B and 3G) or two-way ANOVA (FIGS. 1B, 1D, 2B and 2D) followed by Tukey post hoc. Differences were considered significant at P<0.05. Student's t-test was performed in part of FIGS. 1B and 1E for comparison between two groups.

### Example 2: Daily Administration—Body Weight and Food Intake in DIO Mice

Cohort 1 mice were used to investigate whether combined treatment with semaglutide and a lower dose avasimibe compared to single semaglutide injection in morbidly obese mice (FIG. 1A). On the first day of the experiment, the average body weight of the mice was recorded as 48.84±2.26 grams (FIG. 1B). After four weeks of treatment, mice in the semaglutide and combination groups were sacrificed since their body weight had reached a plateau and was significantly lower than that of mice maintained on a normal diet (data not shown). Mice in the vehicle control and avasimibe groups were sacrificed after additional 16 days of treatment (FIG. 1A). Throughout the initial 4-week period, the mean body weight of the vehicle-treated animals increased by 1.2% compared to their starting body weight. In contrast, the mean body weight changes for mice treated with avasimibe, semaglutide, or the combination of both drugs were −6.29%, −21.66%, and −30.47% of their starting body weight, respectively (FIG. 1C). Notably, low dose avasimibe treatment (10 mg/kg BW) alone led to only marginal body weight loss compared with our previous findings where a 24% reduction in body weight was achieved through daily intraperitoneal (ip) administration of 20 mg/kg BW avasimibe for 14 day. Y. Zhu, S. Q. Kim, Y. Zhang, Q. Liu, K. H. Kim, Pharmacological inhibition of acyl-coenzyme A:cholesterol acyltransferase alleviates obesity and insulin resistance in diet-induced obese mice by regulating food intake, Metabolism 123 (2021) 154861, which is incorporated herein by reference. Consequently, we increased the avasimibe dose to 20 mg/kg BW in the subsequent study involving the second cohort of mice. The observed body weight loss following the administration of semaglutide and the combination treatment could be attributed to rapid suppression of food intake when compared to mice treated with the vehicle or avasimibe alone (FIG. 1D). However, as the treatment regimen continued, food intake gradually returned to baseline levels, and the effect of semaglutide diminished around day 26. Furthermore, measurements of organ weights confirmed a decrease in all collected fat depots in mice from the combination group compared to the semaglutide group (FIG. 1E). While kidney weight did not show any significant differences, the liver and brain weight were notably increased in the combination group compared to the semaglutide group (FIG. 1E). To investigate potential toxic effects of the combination treatment on the liver, we measured plasma ALT and AST levels. Both ALT and AST levels were higher in the combination group compared to the semaglutide group and were comparable to the levels shown in the vehicle control group (Table 1). These results suggest that the repeated sc administration of semaglutide in combination with avasimibe resulted in greater weight reduction, primarily attributed to the loss of fat mass in DIO mice.

TABLE 1

| Plasma | Day 43 | | | Day 29 | | |
|---|---|---|---|---|---|---|
| markers | Vehicle | Avasimibe | p | Semaglutide | Combination | p |
| Glucose | 178.07 ± 13.55 | 139.7 ± 6.32* | 0.0195 | 121.73 ± 6.26 | 91.16 ± 6.64^{§} | 0.0036 |
| (mg/dL) | | | | | | |
| Insulin | 0.94 ± 0.26 | 1.43 ± 0.29 | NS | 0.41 ± 0.07 | 0.26 ± 0.07 | NS |
| (mIU/mL) | | | | | | |
| Leptin | 11.02 ± 1.45 | 6.72 ± 1.40* | 0.0472 | 2.99 ± 0.65 | 0.30 ± 0.12^{§} | 0.0007 |
| (pg/mL) | | | | | | |
| Cholesterol | 9.2606 ± 0.237 | 8.698 ± 0.315 | NS | 8.055 ± 0.292 | 5.979 ± 0.217^{§} | 2.05 × 10⁻⁵ |
| (mg/ml) | | | | | | |
| CE | 7.390 ± 0.204 | 7.126 ± 0.202 | NS | 7.071 ± 0.240 | 5.116 ± 0.178^{§} | 3.66 × 10⁻⁶ |
| (mg/ml) | | | | | | |
| LDL | 8.17 ± 0.62 | 7.14 ± 0.46 | NS | 5.05 ± 0.60 | 4.73 ± 0.34 | NS |
| (mg/dL) | | | | | | |
| HDL | 36.97 ± 2.13 | 35.13 ± 1.14 | NS | 37.02 ± 1.14 | 25.79 ± 0.86^{§} | 1.33 × 10⁻⁶ |
| (mg/dL) | | | | | | |
| TG | 29.40 ± 1.89 | 33.80 ± 2.09 | NS | 21.60 ± 1.38 | 27.70 ± 1.73^{§} | 0.0130 |
| (mg/dL) | | | | | | |
| AST | 64.35 ± 7.37 | 56.87 ± 3.85 | NS | 46.40 ± 2.35 | 70.93 ± 5.79^{§} | 0.0010 |
| (U/L) | | | | | | |
| ALT | 88.46 ± 20.63 | 92.03 ± 13.35 | NS | 32.22 ± 3.60 | 80.59 ± 8.30^{§} | 4.41 × 10⁻⁵ |
| (U/L) | | | | | | |

Values are mean±SEM from n=10 mice/group. (ALT, alanine aminotransferase; AST, aspartate transaminase; CE, cholesteryl ester; HDL, high-density lipoprotein; LDL, low-density lipoprotein; NS, not significant; TG, triglyceride.) For the comparison between two groups that were sacrificed on the same day, the values were analyzed using Student's t-test. * indicates significant difference between the Vehicle group and the Avasimibe group that were sacrificed on D43, and § indicates significant difference between the Semaglutide group and the Combination group.

### Example 3: Daily Administration—Cholesterol and Leptin Level in DIO Mice

To assess whether avasimibe could enhance obesity-associated plasma lipoprotein profiles and major lipid components, we analyzed the plasma samples from mice in Cohort 1. The daily sc administration of 10 mg/kg BW avasimibe over a period of six weeks did not lead to a significant reduction in cholesterol or CE levels when compared to the vehicle treatment (Table 1). However, the combination treatment for four weeks resulted in a significant decrease in both free cholesterol and CE levels compared to the single semaglutide treatment (Table 1). The reduced levels of circulating cholesterol in the combination group were also reflected in the levels of HDL since mice naturally lack CE transfer protein and store most of their cholesterol in HDL. Z. Kaabia, J. Poirier, M. Moughaizel, A. Aguesse, S. Billon-Crossouard, F. Fall, M. Durand, E. Dagher, M. Krempf, M. Croyal, Plasma lipidomic analysis reveals strong similarities between lipid fingerprints in human, hamster and mouse compared to other animal species, Sci Rep. 8 (2018) 15893. Interestingly, avasimibe did not improve triglyceride (TG) levels but rather increased them in the combination group compared to the semaglutide group, reaching levels comparable to the vehicle control (Table 1). Furthermore, the circulating levels of leptin were significantly lower in the combination group compared to the semaglutide group, which is indicative of reduced adiposity in adipose tissue. The avasimibe group in our current study exhibited lower glucose levels compared to the vehicle group, and the combination group showed lower glucose levels compared to the semaglutide group, while the difference in the insulin levels did not reach statistical significance (Table 1). Collectively, the repeated sc administration of semaglutide in combination with avasimibe proved effective in lowering circulating cholesterol and glucose levels without improving TG levels. The reduction in fat mass due to the combination treatment was further supported by the observation of low plasma leptin levels in DIO mice.

### Example 4: Every 3-Day Administration—Body Weight and Food Intake in DIO Mice

To validate the impact of the combined treatment of semaglutide and avasimibe in DIO mice and to simulate a weekly semaglutide regimen for humans, we utilized Cohort 2 mice (FIG. 2A). On day 1, the average body weight of the mice was recorded as 37.37±3.58 grams. Throughout the treatment period, the mice in the vehicle group continued to gain weight, reaching an average body weight of 41.58±4.74 grams, representing a 10.07% increase from the starting body weight (FIG. 2B). In contrast, mice receiving sc administration of avasimibe alone or in combination with semaglutide every three days successfully prevented weight gain induced by HF diet. Specifically, the mean body weight losses were 0.82%, 8.56%, and 14.89% of the starting body weight for the mice treated with avasimibe, semaglutide, and the combination of both drugs, respectively (FIG. 2C). Notably, each administration of semaglutide initially led to a decrease in food intake, which was then recovered within 48 hours. The compensatory effect on food intake became more pronounced by the seventh administration, causing the food intakes of the semaglutide group and the combination group to surpass that of the vehicle group by day 22 (FIG. 2D). However, throughout the entire experiment period, the cumulative food intake analysis revealed that the mice in the combination group consumed less food compared to the mice in the vehicle group (FIG. 2E). Next, we performed an OGTT to validate the suggested improvement in glucose metabolism by avasimibe (FIGS. 2F and 2G). Every three-day sc administration of avasimibe alone did not enhance glucose tolerance nor amplify the hypoglycemic effect of semaglutide. These results suggest that the combination of semaglutide and avasimibe resulted in a greater percentage of body weight loss compared to the effect observed with semaglutide alone in moderately obese mice with little improvement in glucose tolerance.

### Example 5: Every 3-Day Administration—Fat Mass and Adipocyte Size in DIO Mice

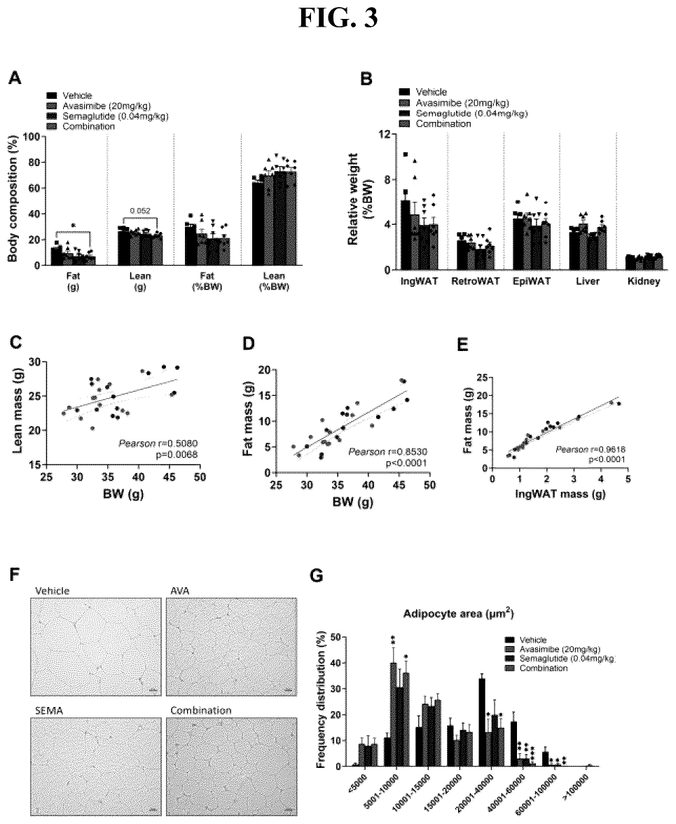

We next tested whether the enhanced body weight loss observed in the combination group was mediated by the reduction in fat mass induced by avasimibe. We found that only the combination group exhibited a significant decrease in fat mass compared to the vehicle group (FIG. 3A). However, when examining the weights of different fat depots, no notable significant differences were observed between the groups due to high variability (FIG. 3B). We further found that fat mass displayed a stronger correlation with body weight than lean mass (FIGS. 3C and 3D). Among the various fat depots, the weight of the inguinal fat depot exhibited the best representation of overall fat mass (FIG. 3E). Since hypertrophic adipocytes are known to exhibit impaired response to insulin and secrete inflammatory cytokines [16-18], we analyzed digital images of H&E-stained inguinal fat pad (FIGS. 3F and 3G). The avasimibe group demonstrated a significantly higher percentage of small adipocytes (5,001-10,000 μm²) and a lower percentage of large adipocytes (20,001-100,000 μm²) compared to the vehicle group. The semaglutide group showed a lower percentage of large adipocytes (40,001-100,000 μm²) compared to the vehicle group. However, no significant changes in adipocyte size distribution were detected in the combination group. Collectively, these findings indicate that the combination treatment reduced fat mass in DIO mice, partially due to avasimibe-induced reduction in fat cell size.

Those skilled in the art will recognize that numerous modifications can be made to the specific implementations described above. The implementations should not be limited to the particular limitations described. Other implementations may be possible.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A method for controlling body weight comprising co-administering to a subject in need a therapeutically effective amount of one or more ACAT inhibitors or pharmaceutically acceptable salts thereof and a therapeutically effective amount of one or more GLP-1 RAs or pharmaceutically acceptable salts thereof.

2. The method according to claim 1, wherein said GLP-1 RA is selected from the group consisting of lixisenatide, liraglutide, exenatide, exenatide extended release, albiglutide, semaglutide, ITCA 650, dulaglutide, tirzepatide, retatrutide, orforglipron, lotiglipron, efpeglenatide, and taspoglutide.

3. The method according to claim 1, wherein said ACAT inhibitor is selected from the group consisting of avasimibe (CI-1011), CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511), E5324, FR145237, CL277,082, YM-17E, FR129169, K-604, pyrocarbonate, beauveriolides I, and methanol extracts of Saururus chinensis root containing saucerneol B and manassantin B.

4. The method according to claim 1, wherein said GLP-1 RA and said ACAT inhibitor are formed in a formulation and the formulation is administered.

5. The method according to claim 1, wherein said GLP-1 RA and said ACAT inhibitor are formulated separately and administered at the same time or sequentially.

6. The method according to claim 1, wherein said GLP-1 RA is selected from the group consisting of semaglutide, liraglutide, and tirzepatide, and said ACAT inhibitor is avasimibe or a pharmaceutically acceptable salt thereof.

7. A pharmaceutical composition comprising:
an ACAT inhibitor or a pharmaceutically acceptable salt thereof;
a GLP-1 RA or a pharmaceutically acceptable salt thereof; and
a pharmaceutically acceptable excipient or carrier.

8. The pharmaceutical composition according to claim 7, wherein said GLP-1 RA is selected from the group consisting of lixisenatide, liraglutide, exenatide, exenatide extended release, albiglutide, semaglutide, ITCA 650, dulaglutide, tirzepatide, retatrutide, orforglipron, lotiglipron, efpeglenatide, and taspoglutide.

9. The pharmaceutical composition according to claim 7, wherein said ACAT inhibitor is selected from the group consisting of avasimibe (CI-1011), CI-976, CP113,818, pactimibe, NTE-122, F-1394, PD140296, PD128042, PD132301-2, octimibate, DuP128, 58-035, HL-004, SMP-500, CL-277,082, SKF-99085, CS-505, eflucimibe (F12511), E5324, FR145237, CL277,082, YM-17E, FR129169, K-604, pyrocarbonate, beauveriolides I, and methanol extracts of Saururus chinensis root containing saucerneol B and manassantin B.

10. The pharmaceutical composition according to claim 7, wherein said GLP-1 RA is selected from the group consisting of semaglutide, liraglutide, and tirzepatide, and said ACAT inhibitor is avasimibe or a pharmaceutically acceptable salt thereof.
